(19) United States
(12) Patent Application Publication
(10) Pub. No.: US 2008/0194902 A1
(43) Pub. Date: Aug. 14, 2008

(54) ADSORBENT FOR DIENES REMOVAL FROM LIQUID AND GAS STREAMS

(76) Inventors: Albert Michael Tsybulevski, Louisville, KY (US); Alexander A. Greish, Moscow (RU); Leonid M. Kustov, Mosco (RU)

Correspondence Address: ALBERT M. TSYBULEVSKI, 3010 KIPLING WAY, LOUISVILLE, KY, 40205, US

(21) Appl. No.: 11/674,019

(22) Filed: Feb. 12, 2007

(51) Int. Cl. C07C 7/12; B01J 20/02; B01J 20/04; B01J 20/16

(52) U.S. Cl. 585/820; 502/406; 502/407; 502/410; 502/411; 977/810

(57) ABSTRACT

It is now possible to purify diene-contaminated liquid and gas streams by treating these streams with adsorbents comprising single or multiple transition metal polycation-exchanged faujasites having silicon to aluminum ratio from 0.9 to about 9.0, wherein said transition metal (Tr) polycations have the general formula [Tr_{α}O_{β}]ⁿ⁺, wherein a varies from 2 to 8, β—from 0 to 4, and n—from 1 to 3 and wherein said transition metals include IB. IIB, VIIB and VIII Group metals preferably selected from the group consisting of copper, cadmium, zinc, manganese, nickel and iron.

### FIELD OF INVENTION

The invention relates to an adsorbent for removal of dienes, including mainly C₄-C₆ diolefins, from liquid and gas streams, and more particularly a novel nano-structured transition metal exchanged zeolite adsorbent for purification of hydrocarbons, monomers, solvents, cracking gases and steam cracking liquids, as well as off-gases from refiners and petrochemical plants, flue and automotive exhaust gases. The invention also relates to processes for the use of the dienes adsorbent for liquid and gas streams purification.

### DESCRIPTION OF PRIOR ART

Dienes are common impurities of many commercial petrochemical streams, particularly of those that originated from steam cracking processes. Due to dienes' strong tendency to polymerization, their presence in process fluids tighten pipes, trays, valves and other equipment. At the same time, dienes are strong poisons for majority of catalysts and adsorbents for petrochemical processes so that dienes' presence in hydrocarbon stocks, monomers, co-monomers, solvents and other petrochemical sources is inadmissible. On the other hand, as stated in G. Dollard's publication (Chemico-Biological Interactions, v. 135-136, 2001, p. 177-206) dienes are common components of many industrial waste gases. Dienes may widely be encountered in flue gases of power stations, industrial and residential burners and flares as well as in gases emitted into the atmosphere by chemical, petrochemical and automotive industries. For instance, a substantial concentration of butadiene was found in combustion products of residential wood fireplaces. It is also a usual pollutant of internal combustion engine (ICE) exhaust gases. Aside from the fact that dienes per se comprise severe health and environmental hazards, they often undergo photochemical conversion in the atmosphere producing more aggressive hazards.

Accordingly, dienes removal from commercial streams and abatement of their emissions into atmosphere is a well-developed field by many inventors. For example, U.S. Pat. Nos. 6,169,218, 6,858,766, 6,977,321 and 7,002,050 employ selective hydrogenation of dienes for purification of olefin-contaminated flows, cracking gas, individual monomers and steam cracking gasoline. The process is carried out at moderate temperatures of 50-250° C., pressure of 20-30 bars and contact times <0.1 sec over platinum group catalysts. Selective hydrogenation requires substantial capital and operation expenses and can be offset for large capacities and in the case of simultaneous removal of acetylene hydrocarbons.

For dienes emission control, alongside other volatile organic compounds (VOC), catalytic oxidation (combustion) technologies have been developed. U.S. Pat. Nos. 6,403,048 and 6,818,195 suggest advanced catalytic systems, which can provide hydrocarbon pollutants abatement at low temperatures, <100° C. However, even the highly efficient complex platinum-metal oxide-zeolite system, which is disclosed in U.S. Pat. No. 6,818,195, in order to reach a practically complete diene pollutant destruction up to 99.9%, requires significant contact times of 0.2-1.0 sec and corresponding volumes of the catalyst bed. Another common demerit of catalytic combustion consists of its non-selectivity and inapplicability to purification of combustible, hydrocarbon-contaminated flows.

Adsorption processes for dienes removal from variety of commercial stocks and emissions are widely used in the industry. Practically all known types of adsorbents were suggested for the purpose. GB Patent No 966,000 describes the use of silica gel for butadiene removal. The main drawback of the system consists of low adsorption capacity in respect to butadiene at its low partial pressure. Thus, the inventors were forced to apply a moving bed or fluid flow technique with continuous adsorbent regeneration.

U.S. Pat. No. 6,987,165 teaches to apply activated carbon, molecular sieves and ion exchangers for 1,3-butadiene removal from C₄-fraction of steam crackers or FCC (fluid catalytic cracking) crackers to reach residual content of impurity on the level of 200-500 ppm. U.S. Pat. No. 4,567,309 demonstrates that activated carbons and particularly carbon molecular sieves (CMS) are characterized by extended adsorption capacity for dienes. Therewith, adsorption of butadiene at its low concentrations (<100 ppm) remains insufficient, so that such an intricate technique as a simulated moving bed has been suggested for 1,3-butadiene separation.

Another popular group of diene adsorbents are synthetic zeolites or molecular sieves. U.S. Pat. No. 3,992,471 discloses sodium- and potassium-exchanged forms of zeolite X as adsorbents for butadiene removal from gas and liquid streams. Although molecular sieve 13× is currently the most prevalent commercial adsorbent for dienes, a low adsorption capacity of this adsorbent at low diene partial pressures and concentrations cannot satisfy contemporary requirements for petrochemical stocks purity.

Several attempts to overcome this generic disadvantage of molecular sieve adsorbents were undertaken. Arruebo, “Separation of Binary C₅ and C₆ Hydrocarbon Mixtures through MFI Zeolite Membranes” (J. of Membrane Science, vol. 269, No 1-2, 2006, p. 171-176) describes the use of B-ZSM-5 membrane for pentane/isoprene separation. At the same time, Badani, “Effects of Cs and Cl on Butadiene Adsorption on Ag/α-Al₂O₃ Catalysts” (J. of Catalysis, vol. 206, No 1, 2002, p. 29-39) utilizes chemisorption over alumina-supported silver catalyst for dienes adsorption enhancement. Indeed, double bonds interaction with dispersed silver leads to significant increase of dienes recovery degree. But chemisorption is an irreversible process and requires oxidizing regeneration of the catalyst.

Substantial improvement of butadiene adsorbents was achieved. in U.S. Pat. Nos. 6,215,037 and 6,911,569, which apply a concept of π-complexation of double bonds with silver and copper cations incorporated into zeolite structure. The main advantage of the proposed adsorbents is a high selectivity of dienes adsorption versus monolefins. This property is very valuable for several petrochemical applications. The disclosed efficient adsorbents are Ag(I)- and Cu(II)-exchanged faujasites X and Y with an unusually high ion exchange degree on the level of 95-97% equiv. In addition, Takahashi, “Cu(I)—Y-Zeolite as a Superior Adsorbent for Diene/Olefin Separation” (Langmuir, vol. 17, No 26, 2001, p. 8405-8413) uses monovalent copper-exchanged zeolite Y for diene separation from olefins.

The main disadvantages of the disclosed zeolite systems include, apart from high cost, a low stability and readiness to undergo oxidation/reduction. They are also difficult to manufacture even in a pilot plant, not to mention on a commercial scale. Cu(I)-salts are insoluble in water, and this demands the use of a complex technique for ion exchange procedure. On the other hand, enormous, close to equilibrium Cu and Ag ion exchange degrees, which are specified in U.S. Pat. No. 6,215,037, can be reached only by means of multiple exchange operations, which in turn result in a long chain of ion exchange columns and huge overexpenditure of metal salt solutions. Moreover, adsorption capacity of the obtained Ag-, Cu-zeolite adsorbents appeared to be insufficient at low butadiene partial pressures, so that the inventors were forced to additionally incorporate zeolite CaA into their adsorbent formulation for preliminary adsorbate (butadiene) concentration.

It is also important that most of the prior art diene adsorbents are highly hydrophilic materials, so that water adsorption may completely suppress diene adsorption. This particularly affects petrochemical and waste gases treatment applications, when moisture content significantly exceeds diene concentration. As a result, such adsorbents require a supplemental desiccant bed for preliminary deep dehydration of gas and liquid streams.

Even though most of the prior art adsorbents have been applicable for dienes removal from liquid and gas streams, it is expedient to develop a principally novel adsorbent, which would be deprived of the disadvantages pointed above.

Accordingly, it is an aspect of our invention to provide an adsorbent for liquid and gas streams purification, having enhanced adsorption capacity at diene low partial pressures and concentrations. Other substantial objects and advantages of the present invention include:
to provide a low-cost, easily accessible and stable adsorbent for dienes removal;
to provide an adsorbent, which is capable to reach a residual content of dienes in purified liquid streams on the level of 500 parts per billion (ppb) and in purified gas streams on the level below 1,500 ppb;
to provide an adsorbent, which would be suitable for purification of heavily moisturized liquid and gas flows;
to provide an adsorbent having a high selectivity in respect to dienes at conditions of strong competitive adsorption of other adsorbable substances such as olefins, sulfur and nitrogen-contaminated compounds;
to provide an adsorbent which would be applicable for purification of commercial waste flows, flue and automotive exhaust gases.

It is a further object of the invention to reveal a simple and reliable process for dienes removal from liquid and gas streams using the adsorbent of the present invention, which will be capable of providing a gas and liquid stream purity outlet the adsorbent bed significantly higher than the presently available level of residual diene content of 2-5 ppm. These and still further objects and advantages of the invention will become apparent from ensuing description of a preferred embodiment of the invention and examples therewith.

### SUMMARY OF INVENTION

In accordance with the present invention, an adsorbent for removing dienes from liquid and gas streams, which manifests enhanced adsorption capacity particularly at very low diene concentrations and partial pressures, is provided. The adsorbent comprises synthetic faujasites LSF, X and Y with a silicon to aluminum ratio from about 0.9 to about 9.0, wherein mono- and polynuclear transition metal (TrM) cations are incorporated into the synthetic faujasites structure, and wherein said transition metals are selected from Group IB, IIB, VIIB and VIII of the Periodic Table, preferably selected from group of mono-, bi- and trivalent cations of copper, zinc, cadmium, manganese, nickel, and iron. Said polynuclear transition metal cations have a nanometer range size, from about 0.4 nm to about 1.6 nm, and a general formula [TrM_{α}O_{β}]ⁿ⁺, wherein a varies from 2 to 8, β—from 0 to 4, n—from 1 to 3, and content of said polycations in the faujasites structure comprises from 1.5 to 12% w.

The present invention also features a process for purifying diene-contaminated liquid and gas streams, which comprises passing said liquid or gas stream over the bed of an adsorbent and intermittent regenerating said adsorbent in a hydrogen-contaminated gas flow at a temperature from about 150 to about 200° C.

### DRAWING FIGURES

FIG. 1 shows isotherms of 1,3-butadiene adsorption from hexene-1 solution over transition metal polycation exchanged faujasite adsorbents of the invention and Cu monocation exchanged LSF adsorbent of the prior art;

FIG. 2 compares chromatograms of initial nitrogen-propylene-butadiene mixture (b) and purified product over adsorbent of Example 8 of the present invention (a);

FIG. 3 demonstrates gas analyses at 11^{th} pulse of combustion products purification over adsorbent of Example 11 of the present invention (a) in comparison with a chromatogram of initial gas mixture (b).

### DETAILED DESCRIPTION OF THE INVENTION

The use of the sodium form of synthetic faujasite X for the adsorption of dienes from liquid and gas streams is known. It is also known that a practically complete substitution of alkali metal cations by copper or silver cations leads to an increase of the conventional faujasites adsorption capacity in respect to dienes. It has now surprisingly been discovered by the authors of the present invention that faujasites, which structure includes polynuclear transition metal cations selected from the group of copper, zinc, cadmium, manganese, nickel and iron cations, significantly overperform in dienes adsorption mononuclear copper and silver cation-exchanged faujasites disclosed in U.S. Pat. Nos. 6,215,037 and 6,911,569. Thus, an introduction of transition metal polycations into the faujasite structure constitutes the most essential improvement of the prior art adsorbents for dienes removal.

It became known in the last ten years that zeolite intracrystalline cages could be used for alkali and TrM polycations stabilization. Direct evidence of transition metal and their oxide cluster polycations formation in the respective ion-exchanged forms of molecular sieves A, X, and Y is presented, for example in the following publications:
[1] “Zinc Oxide Nano-Cluster Formation in Zeolite” by Hoo Bum Lee et al., Bulletin of Korean Chemical Society, vol. 19 (No 24), 1998, pp. 1002-1005;
[2] “Zeolites as Matrices for the Stabilization of Unusual Cationic Zinc Species” by F. Rittner et al., Microporous and Macroporous Materials, vol. 24, (No 4-6), 1998, pp. 127-131;
[3] “UV/VIS Diffuse Reflectance Study on the Formation of Zinc Oxide Clusters in Zeolites” by M. Wark et al., Bulgarian Chemical Communications, vol. 30 (No 1-4), 1998, pp. 129-142;
[4] “Characterization of Zinc Oxide Nanoparticles Encapsulated into Zeolite-Y” by F. Meneau et al., Nuclear Instruments and Methods in Physics Research, Section B, vol. 199, 2003, pp. 499-503;
[5] “Theoretical Analysis of Oxygen-Bridged Cu Pairs in Cu-Exchanged Zeolites”, by B. Goodman et al., Catalytic Letters, vol. 56 (No 4), 1999, pp. 183-188.

Such cluster metal or metal oxide polycations have the general formula [TrM_{α}O_{β}]²⁺, where α varies from 2 to 8 and β varies from 0 to 4. Accordingly, cluster polycations have diameters in nano- or sub-nanometer range of 4-16 Å or 0.4-1.6 nm, which determine an enormous value of metal component surface area on the level of 1000-4000 m²/g. The practical application of zinc polycation-exchanged low-silica faujasite is found in U.S. Pat. No. 6,096,194, which describes an adsorbent for sulfur-contaminated compounds having an extremely high adsorption capacity.

The disclosed methodologies for the synthesis of TrM polycations use a chemical vapor deposition (CVD) technique, such as in the above cited references [1] and [2], or alkaline treatment of conventional exchanged zeolites, such as in the cited references [3] and [4]. Both procedures are suitable for lab synthesis and are extremely hard and expensive to implement even in a pilot plant, not to mention on a commercial scale. At the same time, U.S. Pat. No. 6,096,194 teaches that zinc cluster polycations can be easily synthesized in the presence of partially hydrolyzed or reduced cations.

It has been found in the present invention that this hydrolytic methodology might be successfully expanded for obtaining other transition metal polycation-exchanged faujasites, including copper, cadmium, manganese, nickel and iron. Generally, formation of the cluster polycations from hydrolyzed mononuclear transition metal cations can be expressed, for example for bivalent cations, by the following reactions:

2[TrM(OH)]⁺→[TrM₂O]²⁺+H₂O (1)

[TrM(OH)]⁺+TrM²⁺→[TrM₂O]²⁺+H⁺ (2)

Alternatively, polycations of [TrM₂]²⁺ type may be formed by the condensation reaction of mononuclear metal ions with zero-valent metal atoms as follows:

TrM²⁺+TrM⁰→[(TrM₂]²⁺ (3)

The simultaneous occurrence of the reactions (1)-(3) and the consecutive evolution of the reactions (2) or (3) leads to the formation of cluster polycations of the above mentioned general formula [TrM_{α}O_{β}]²⁺.

It was also discovered in the present invention that the materials, which are synthesized in the described process, are characterized by a high dispersity of transition metal oxide clusters (0.4-1.6 nm size) and their uniform distribution in zeolite crystals. Accordingly, a high adsorption activity of TrM polycation exchanged zeolites for dienes removal was demonstrated also. Indeed, transition metals and their oxides readily react with double bonds, so that dienes chemisorption over metal surface usually comprises the first step of their catalytic hydrogenation Thus, changeover bulk crystal metals in the conventional catalysts into nano-sized phase in the newly invented polycation adsorbents, along with enhancement of metal component surface area, must increase its reactivity and simultaneously weaken activation energy of dienes chemisorption. In such a manner, it appears to be possible to obtain efficient, reversible chemisorbents for dienes removal.

As this takes place, it was established that a significant level of dienes adsorption can be attained at the total TrM cation exchange degree in the range of 60-80% (equiv.). And what is more, it has now surprisingly been found that a superior diene adsorption capacity of TrM-exchanged faujasites depends not on a total content of mono- and polynuclear TrM cations but mainly on the portion which [TrM_{α}O_{β}]²⁺.polycations comprise in the total ion exchange degree. In such a manner, a proper adsorbent for dienes removal from liquid and gas streams must include at least 3.5% (equiv.) of TrM polycations, so that their content in the adsorbent composition may comprise from about 1.5 to about 12% w.

The balance of the cations in the faujasite structure are alkali and/or alkaline-earth metals selected preferably from the group of sodium, potassium, magnesium, and calcium. The content of balance cations, for example at the preferred TrM cation exchange degree in the range of 70-80% (equiv.), may comprise correspondingly 20-30% (equiv.).

Therefore, an adsorbent for dienes removal, according to the present invention, is to be characterized by the general formula:

(Tr₁M⁺)ₘ[Tr₁M_{α}O_{β}]ₙ⁺(Tr₂M²⁺)ₚ[Tr₂M²⁺_{γ}O_{δ}]_{q}²⁺(Tr₃M)_{c}[Tr₃M_{ε}O_{ν}]_{d}(Me⁺)ₑ(Me²⁺)_{f}Si_{σ}Al_{τ}O_{φ},

where:
Tr₁M, Tr₂M, Tr₃M are mononuclear mono-, bi-, and trivalent TrM cations,
[Tr₁M_{α}O_{β}]⁺, [Tr₂M_{γ}O_{δ}]²⁺, [Tr₃M_{ε}O_{ν}], are corresponding polycations;
n, q and d are Tr₁M, Tr₂M and Tr₃M polycation exchange degrees;
α, γ and ε are numbers of TrM atoms in polycations that vary from 2 to 8;
β, δ, and ν are numbers of oxygen atoms in TrM polycations;
Me⁺, Me²⁺ are monocations of alkali and alkaline-earth metals;
m, p, c, e, f are corresponding ion exchange degrees;
σ/τ is silicon to aluminum ratio that varies from 0.9 to 9.0.

Understandably, polycation exchanged zeolites contain an inequivalent excess of Tr₁M, Tr₂M and Tr₃M cations, so that the ratio of the sum of all cation equivalents to the aluminum equivalent,

$$Ψ = \frac{m + n + p + c + d + q + e + f}{τ} \geq 1.05$$

and preferably is above 1.20 in order to provide the total content of TrM polycation nano-clusters from about 1.5 to about 12% w.

On the whole, the adsorbents of the present invention, in accordance with the designated general formula, are prepared by means of a conventional ion exchange process using the alkaline (sodium and/or potassium) forms of synthetic faujasite powders or granules as the starting material. In so doing, an ion exchange is carried out with water solutions of transition metal salts, preferably chlorides, nitrates, sulfates, acetates, etc. In order to achieve TrM polycations formation in the faujasites structure, the specific conditions for partial hydrolysis of TrM salts are maintained over a complete ion exchange process.

For example, the adsorbent, according to the present invention can be produced by means of treating alkaline forms of faujasites with very dilute 0.005-0.1 N TrM salt solutions, maintaining pH of zeolite/solution medium in the range of 5.5-6.5. Alternatively, a required content of TrM polycations in faujasite structure is to be furnished employing concentrated 0.5-5 N TrM salt solutions at a pH in the range of 5.0-6.2 controlled by an appropriate buffer. A preliminary ion exchange of alkali metal cations with alkaline-earth metal cations may be helpful for maintaining pH in the desired range over the whole cation exchange process. As this takes place, a suitable ion exchange degree for alkaline-earth metals may be in the range of 30-60% (equiv.).

Accordingly, the adsorbent of the present invention is an easily accessible and economical to manufacture product.

Incorporation of TrM polycations into the faujasite structure creates a product, which is particularly useful for dienes removal from various liquid and gas streams. Appropriate applications for the dienes adsorbent, in accordance to the present invention, include: cracking gas, ethylene and propylene monomers, hexene-1 and octene-1 co-monomers purification, isobutylene and C₄ cuts from steam crackers or FCC (fluid catalytic cracking), or isobutane dehydrogenation in the MTBE (methyl tert-butyl ether) production, butane/butene (BB) and pentane/pentene (PP) fractions in the alkylation process. The adsorbent of the present invention is also useful for purification of automotive exhaust gases, to protect the catalyst from early coking and deactivation, and for rendering harmless miscellaneous flue gases and off-gases from synthetic rubber and petrochemical plants.

An advantageous application of the adsorbent constitutes heavily moisturized streams, particularly combustion products of organic material wastes from industrial processes.

It has been discovered that TrM polycation faujasites, due to a nano-sized structure of their active component, weekly chemisorb diolefins, and can be regenerated from diene adsorbate at comparatively mild desorption conditions, which prevent adsorbed dienes oligomerization and extensive coking of the adsorbent. One of suitable procedures for the adsorbent regeneration consists of rinsing the adsorbent bed with about ⅛- 1/10 portion of a purified flow at the temperature of diene adsorption and heating the adsorbent bed in a hydrogen or hydrogen-contaminated gas flow at 150-200° C.

As it was demonstrated in the example, the total coke accumulation in the adsorbent after 30 adsorption/regeneration cycles does not exceed 0.03% w., when the above-described regeneration procedure is employed. Such low level of the adsorbent coking assures a long-term, continuous and economical exploitation of the adsorbent and purification process using the adsorbent, according to the present invention.

The process for purification of liquid or liquefied streams, for example propylene, hexene-1, isobutene, cracking liquid, C₄-cuts or BB/PP fractions, consists of passing those streams through the bed of adsorbent of the present invention under the following conditions: temperature—from about 0 to about 60° C., pressure—from atmospheric to about 60 bars, and LHSV (liquid hourly space velocity)—from 0.1 to 10 h⁻¹. The purification process can be conducted as long as there are diene traces appearing in the liquid product outlet the adsorbent bed. At that point, the adsorbent, which is loaded with dienes and possibly other co-adsorbed substances, such as moisture, sulfur- and nitrogen-contaminated compounds, is switched to the regeneration step of the process cycle. The adsorbent bed regeneration is conducted as it was described above.

As a regeneration agent, hydrogen or hydrogen-contaminated gases, for example from catalytic reforming or platforming units, as well as methane-hydrogen faction from a cracking gas fractionation unit may be used.

It has been surprisingly established that the adsorbent, according to the present invention, when applied for a hydrocarbon liquids purification process, is capable of reducing dienes content in the product from initial range of 10-500 ppm to the range of 200-500 ppb, a level unavailable to conventional diene adsorbents of the prior art. The process of the present invention also overperforms the selective hydrogenation process in respect to an achievable purity of the product.

Thus, the use of the adsorbent of the present invention and purification process therewith produce a significant commercial effect providing a protection of expensive single-site polymerization catalysts and finally, improvement of polymer product quality. As applied in isobutylene, cracking liquid, C₄-cuts and BB/PP fractions purification, an enhanced purity of the feed warrants an appreciable reduction of the catalyst expense in alkylate and MTBE manufacturing processes.

Similar advantages are to be accomplished at the use of the adsorbent and process, according to the present invention, for purification of various gas streams, for example cracking gases from steam crackers and FCC. Particularly beneficial is the use of the adsorbent and the process therewith for dienes removal from off-gases from refiners and petrochemical plants, flue gases of power stations and automotive exhaust gases. The preferred conditions for gas stream purification process are: temperature—from 0 to about 100° C., pressure—from atmospheric to about 150 bars, linear velocity—from 0.01 to about 0.4 m/sec. It has also been established that the adsorbent and process, according to the present invention, can provide a residual content of dienes in a gas flow outlet a purification process adsorber on the level of 500-1,500 ppb. Dienes belong to highly toxic substances, so that a significant reduction of their emissions into the atmosphere, which is achievable in an economical way with the use of the present invention, will improve sanitary conditions and environment as a whole around industrial sites.

In order to further illustrate the present invention and its advantages the following examples are presented. Clearly, the examples bear a demonstrative character and cannot show any limitations on the scope of the invention.

### EXAMPLES 1 to 5

### According to the Invention—Single TrM Polycation-Exchanged Faujasites

Original materials, beaded sodium-potassium form of low-silica faujasite (LSF) with Si:Al (M) ratio of 1.01, standard zeolite 13× (M=1.25) and sodium form of synthetic faujasite Y, NaY (M=2.4) were obtained from Zeochem and W. R. Grace. In Examples (1) and (2), 100 g of NaKLSF were treated at constant agitation with 1 L of a 1N water solution of zinc (Example 1) and manganese (Example 2) chlorides. In Examples (3) and (4), 100 g of 13× molecular sieve were treated with 1 L of a 1N solutions of copper chloride (Example 3) and cadmium nitrate (Example 4). And in Example 5, 100 g of zeolite NaY were treated with 1 L of 1N copper acetate solution. In order to maintain the pH of the solutions in the range of 5.6-6.2, in Examples (1)-(4), a phosphate buffer 0.0297 M solution of basic potassium dihydrophosphate-K₂HPO₄, and in Example 5, an acetate buffer, 0.05 M of sodium acetate+0.03 M solution of acetic acid were used at salt solutions preparation and over full 4 hours of the ion exchange operation.

The exchanged products were then washed with deionized water (DIW), dried at 110° C. for 3-4 hours and calcined at 250 for 2 hours. The chemical analysis of the prepared samples was carried out by Atomic Spectroscopy (AA) and/or Inductively Coupled Plasma Atomic Emission Spectroscopy (ICP). The obtained samples have the following cation composition (herein and in all ensuing Examples, polycations and nano-structured transition metal polycation forms of zeolites are marked with index “p” under corresponding cation symbol):

### EXAMPLE 1

(Zn)ₚLSF: Zn—77; Na—27; K—4% (equiv.);

In accordance with the expression Ψ for cation balance of the above-designated formula for polycation-exchanged TrM faujasites, zinc polycations (Zn)ₚ content comprises 4% (equiv.) or 4.1% w.

### EXAMPLE 2

(Mn)ₚLSF: Mn—71; Na—33; K—6; Ca—1% (equiv.). (Mn)ₚ—5.5% (equiv.), or 2.7% w.

### EXAMPLE 3

(Mn)ₚX: Mn—80; Na—37; K—1% (equiv.). (Mn)ₚ—9% or 4.0% w.

### EXAMPLE 4

(Cd)ₚX: Cd—68; Na—39% (equiv.); (Cd)ₚ—3.5% (equiv.) or 2.4% w.

### EXAMPLE 5

(Cu)ₚY: Cu—76; Na—33% (equiv.); (Cu)ₚ—4.5% (equiv.) or 1.5% w.

### EXAMPLE 6

### Comparative

The example relates to reproduction of one of π-complexation adsorbents Cu(97)LSF according to U.S. Pat. No. 6,215,037. Original material NaKLSF zeolite was the same as in Examples 1 and 2. In order to reach >97% (equiv.) Cu²⁼ ion exchange degree, zeolite material was treated four times one after another by copper chloride solutions with a successive increase of the ratio of copper equivalents in solution to sodium-potassium equivalents in zeolite. In so doing, the concentration of CuCl₂ solutions was 1N, 2N, 4N and 6N respectively. The final product has the following cation composition:
Cu—97.4; Na—2.2; K—0.4% (equiv.)

Accordingly, due to low pH range (3.9-4.5) of copper chloride solutions over ion exchange process, there is no hydrolysis and correspondingly no excess of cations, no polycations formation, so that, as claimed in U.S. Pat. No. 6,215,037, the prior art adsorbent contains only copper monocations.

### EXAMPLES 7 TO 9

### According to the Invention—Double TRM Polycation-Exchanged Faujasites)

Starting material NaKLSF was first converted into calcium form. For this purpose, 400 g of zeolite beads were treated with 4 L of 1 N solution of calcium chloride at ambient temperature, pH 6.5-7.0 and continuous agitation over 3 hours. After washing the Ca-exchanged zeolite with 25 L of DIW, the obtained product was treated with 5 L of 1 N copper chloride solution at room temperature over 4 hours keeping pH of the exchanged solution in the range of 5.0-5.4 by means of 0.05 M buffer solution of basic sodium dihydrophosphate (NaH₂PO₄). Obtained in such a manner and properly washed CuCaLSF zeolite was then divided into three portions:

In Example 7 one portion of CuCaLSF was treated with 1 L of 1.5 N solution of zinc chloride over 4 hours at control pH in the range of 5.6-6.0. The applied buffer was 0.03 M solution of basic potassium hydrophosphate (K₂HPO₄).

In Example 8 the second portion of CuCaLSF was treated with 1 L of 1.8 N solution of manganese chloride. Ion exchange conditions and continuance were the same as in Example 7.

In Example 9 the last portion of CuCaLSF was treated with 1 L of 2 N solution of nickel nitrate with control pH of the exchanged solution at the level of 5.0-5.2 over full 4 hours of the ion exchange operation.

All three samples were washed by DIW to reach a negative reaction for chloride and nitrate ions, then cured overnight, dried and calcined as in Examples 1-5. The final products have the following cation composition:

### EXAMPLE 7

(Cu)ₚ(Zn)ₚCaLSF: Cu—59; Zn—28; Ca—27; Na—7; K—1% (equiv.);

Σ (Cu)ₚ, (Zn)ₚ 11% (equiv.) or 5.4% (w.)

### EXAMPLE 8

(Cu)ₚ(Mn)ₚCaLSF: Cu—62; Mn—33; Ca—25; Na—8; K—0.8% (equiv.)

Σ (Cu)ₚ, (Mn)ₚ=14.5% (equiv.) or 7% (w.)

### EXAMPLE 9

(Cu)ₚ(Ni)ₚCaLSF: Cu—71; Ni—20; Ca—28; Na—6; K—1.2% (equiv.)

Σ (Cu)ₚ, (Ni)ₚ=13% (equiv.) or 6.2% (w.)

### EXAMPLE 10

### According to the Invention—Triple TRM Polycation-Exchanged Faujasite

100 g of the starting material NaKLSF zeolite, the same as in Examples 1, 2 and 7-9 was treated with 1 L of 1 N solution of iron(III) chloride over 4 hours at ambient temperature and pH control in the range of 5.2-5.4. The washed product Fe(III)LSF was exchanged with 1 L of 1.5 N solution of zinc chloride, following procedure described in Example 7, and then with 1 L of 2 N solution of manganese chloride as it was described in Example 2. After washing, drying and calcining the final product (Fe)ₚ(Zn)ₚ(Mn)ₚLSF has the following cation composition:

Fe(III)—73; Zn—31; Mn—28; Na—3.5; K—0.8% (equiv.). Total (Fe)ₚ, (Zn)ₚ, (Mn)ₚ polycations content makes up 18% (equiv.) or 12% w.

### EXAMPLES 11 AND 12

### According to the Invention—Zinc Polycation-Exchanged Dealuminated Faujasites

The powder of ultrastable faujasite Y (USY) with silicon to aluminum ratio ˜3.0 in the amount of 400 g was treated with 10 L of 0.05 N solution of Na₂EDTA (disodium salt of ethylenediaminetetraacetic acid at 45° C. for 24 hours. After filtrating and washing the obtained dealuminated powder (DAY) was divided into two even portions.

In Example 11, one portion was converted to sodium form by treating with 2 L of 2 N sodium chloride solution in a beaker at 60° C. and continuous agitation over 4 hours. Obtained in such a manner NaDAY powder was then exchanged with 2 L of 0.75 N zinc chloride solution providing formation of nano-sized zinc polycations in a narrow range of pH (5.6-6.0) over the entire ion exchange operation. In so doing, pH of the exchanged solution was adjusted by 0.0297 M solution of basic potassium dihydrophosphate.

In Example 12, the second 200 g portion of DAY was further dealuminated with 5 L of 0.1 N solution of Na₂EDTA at 60° C. over 24 hours. The produced material was then in succession exchanged with sodium and zinc salt solutions as it was described earlier in Example 11.

Both dealuminated powders of Examples 11 and 12 were properly washed from Cl⁻¹ ions, dried at 110° C. and blended with 20% w. attapulgite binder to form the adsorbent extrudates of 1/16″ size. ICP analysis of the prepared adsorbents produce the following results:

### EXAMPLE 11

(Zn)ₚDAY-3.8.

Si: Al=3.81; Cation exchange degrees: Zn—65.6; Na—35; H⁺—17.3; (Zn)ₚ—12.1% (equiv.) or 2.85% w.

### EXAMPLE 12

(Zn)ₚDAY—5.7.

Si:Al=5.70; Cation exchange degrees: Zn—76.5; Na—29; H⁺—20.3; (Zn)ₚ—12.8% (equiv.) or 2.2% w.

### EXAMPLE 13

### Adsorption Equilibrium Test—Isotherms of Adsorption

Adsorbents according to the present invention: (Zn)ₚLSF of Example 1, (Mn)ₚX of Example 3, (Cu)ₚ(Mn)ₚLSF of Example 8 and (Zn)ₚDAY—5.7 of Example 12 along with the adsorbent Cu(97)LSF of the prior art (U.S. Pat. No. 6,215,037) were tested in 1,3-butadiene adsorption from its solution in hexene-1. Isotherms of butadiene adsorption over the samples were measured using the following methodology:

A portion of the adsorbent was placed in a glass container with 100-500 ml of the stock solution. The stock solutions of butadiene in hexene-1 with concentration in the range of 300 ppb-50 ppm were prepared employing microsyringes and measuring flask dilution technique. The volume ratio solution/portion was taken every time so that the difference between initial and equilibrium butadiene concentration in the solution would not exceed 20% of the initial value. The mixture was maintained at ambient temperature for 2-3 days with intermittent shaking until the concentration of the contaminant reached a constant value. Analysis of stock and research solutions was carried out by a gas chromatograph with a flame ionization detector (FID) and 30 m capillary column with DB-WAX stationary liquid phase.

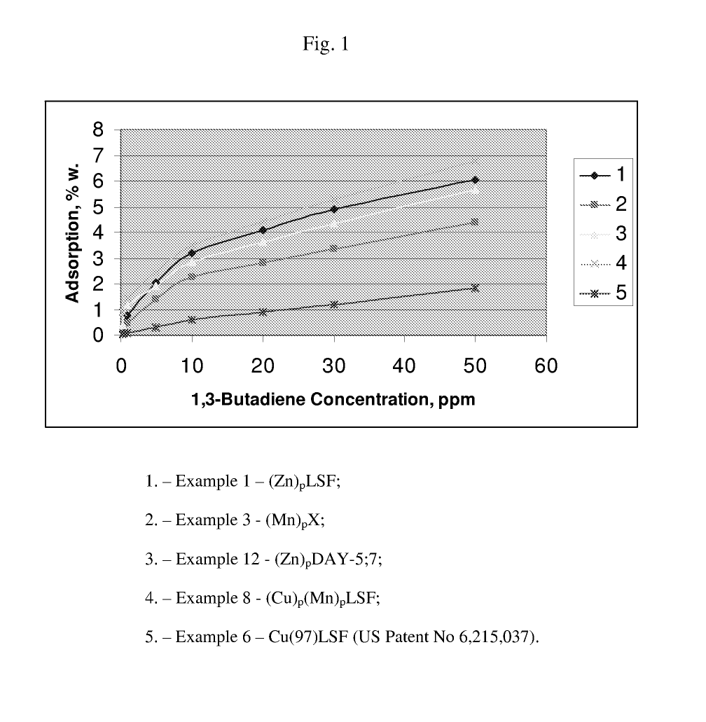

The obtained adsorption isotherms presented in FIG. 1. As it can be seen from the data, all transition metal-exchanged zeolites demonstrate a superior ability to adsorb butadiene at its low concentration in solution. Therewith, nano-structured transition metal polycation adsorbents, which are the subject of the present invention, 3-5 times exceed copper monocation Cu(97)LSF π-complexation adsorbent that is suggested in U.S. Pat. No. 6,215,037.

By themselves, butadiene adsorption values on the level of 2-6% w., which can be reached with the use of adsorbents of the invention at adsorbate concentration range of 0-50 ppm, are absolutely incomparable. And it is remarkable that butadiene adsorption occurred at the conditions of strong competition from hexene-1 side. This feature of the invention makes nano-structured polycation adsorbents particularly valuable for numerous petrochemical applications.

### EXAMPLE 14

### Dynamic Test—Liquid Stream Purification

Adsorbents, according to the invention, (Mn)ₚLSF of Example 2, (Cd)ₚX of Example 4 and (Cu)ₚ(Zn)ₚCaLSF of Example 7, along with the prior art adsorbent, i.e. mono copper cation-exchanged faujasite of Example 6, were tested for 1,3-butadiene removal from hexene-1 flow in a tube adsorber at ambient temperature. The adsorbent bed volume was 15 cm³, diene initial concentration—50 ppm, feed space velocity—10 h⁻¹.

Liquid samples outlet adsorber were taken every 15 min. and analyzed as it was described in Example 13. Thus, 1,3-butadiene breakthrough concentration for each tested adsorbent has been determined, while time before diene breakthrough allows calculation of the samples dynamic adsorption capacity. The data for achieved impurity breakthrough concentrations and adsorption capacities for all tested adsorbents are given in Table 1.

TABLE 1

| | | Dynamic |
|---|---|---|
| Adsorbent | Breakthrough, ppb | capacity, % w. |
| Example 2 | 350 | 2.1 |
| Example 4 | 800 | 1.0 |
| Example 7 | 190 | 3.3 |
| Example 6 | 4,800 | 0.56 |
| (U.S. Pat. No. 6,215,037) | | |

The results of Table 1 demonstrate that, as compared to the adsorbent of the prior art, the present invention provides a profound purity of the product which reaches entirely distinguished technical level. Accordingly, such upgraded feed purity will have a favorable effect on quality of petrochemical products, reduction of catalyst and reagent expenses as well as reduction of other operational costs that are associated with hydrocarbons handling, separation, storage and transportation.

At the same time, enhanced dynamic adsorption capacity of the adsorbents of the present invention permits 2-5 times reduction of required volume of the adsorbers with corresponding decrease of capital investments for purification units construction.

### EXAMPLE 15

### Equilibrium Test—Effect of Moisture

Experimental procedure of Example 13 was repeated in order to determine capability of nano-structured transition metal faujasite adsorbents for dienes removal from moisturized mediums. Adsorbent (Zn)ₚLSF of Example 1, (Cu)ₚY of Example 5, (Cu)ₚ(Mn)ₚLSF of Example 8, (Zn)pDAY of Examples 11 and 12 were tested at ambient temperature for 1,3-butadiene adsorption from dry and moisturized hexene-1 solution with water content of 100 ppm. Butadiene concentration—25 ppm. The results are disclosed in Table 2.

TABLE 2

1,3-Butadiene Adsorption Capacity, % w.

| Adsorbent | From dry hexene-1 | H₂O content - 100-pm |
|---|---|---|
| Example 1 | 4.65 | 2.20 |
| Example 5 | 2.94 | 2.12 |
| Example 8 | 4.80 | 2.30 |
| Example 11 | 3.95 | 3.75 |
| Example 12 | 3.60 | 3.58 |

Table 2 shows that moisture significantly affects ability to adsorb butadiene for adsorbents with a low silicon to aluminum ratio. In that case, diene adsorption on low-silica faujasites (Examples 1 and 8) reveals two-fold diminishing in the presence of 100 ppm water. It should be emphasized therewith that even at such conditions of strong competitive adsorption of water, butadiene adsorption over adsorbents of the present invention, at least twice surpasses capacity of the adsorbent of the prior art in respect to adsorption from dry solution (compare FIG. 1 and Table 2 data).

Meantime, Table 2 demonstrates that diene adsorption over Y-type faujasites, particularly over polycation transition metal-exchanged dealuminated faujasites of Examples 11 and 12 is not more than slightly affected by moisture. This notable feature of polycation-exchanged faujasites of the invention makes their application for many petrochemical processes particularly beneficial.

### EXAMPLE 16

### Equilibrium Test—Effect of Sulfur- and Nitrogen-Contaminated Compounds

Once again the experimental procedure of Example 13 was used to verify ability of the adsorbents of the invention to remove dienes from hydrocarbons polluted by perceptible amounts of organic sulfur and nitrogen-contaminated compounds. Adsorbents, according to the invention, (Mn)ₚLSF of Example 2, (Mn)ₚX of Example 3, (Cu)ₚ(Ni)ₚCaLSF of Example 9 and (Fe)ₚ(Zn)ₚ(Mn)ₚLSF of Example 10 along with the adsorbent Cu(97)LSF of the prior art (Example 6) were used for comparative adsorption of piperylene (1,3-pentadiene) at its concentration of 25 ppm from plain n-pentane and in the presence of dimethyl disulfide (DMDS) and acetonitrile (CH₃CN). In this case, content of impurities comprised: DMDS—50 ppm; CH₃CN—50 ppm. Diene adsorption capacities from plain and polluted n-pentane are compared in Table 3.

TABLE 3

| | Piperylene Adsorption Capacity, % w. | |
|---|---|---|
| | | In presence |
| Adsorbent | Plain n-Pentane | of DMDS and CH₃CN |
| Example 2 | 4.35 | 2.08 |
| Example 3 | 3.37 | 1.85 |
| Example 9 | 4.90 | 4.56 |
| Example 10 | 5.20 | 5.09 |
| Example 6 | 1.65 | 0.44 |
| (U.S. Pat. No. 6,215,037) | | |

As compared to the adsorbent of the prior art, all adsorbents of the present invention demonstrate an appreciably lower sensitivity to the conditions of competitive adsorption of sulfur and nitrogen-contaminated compounds. At the same time, diene adsorption over single transition metal polycation exchanged faujasites of Example 2 and 3 is ˜2 times reduced by traces of DMDS and acetonitrile in the solvent, whereas the ability to adsorb dienes for double and triple transition metal polycation adsorbents of Examples 9 and 10 is practically not affected by organic sulfur and nitrogen-contaminated impurities. Thus, the test results point to one more commercially important merit of the adsorbents of the invention.

### EXAMPLE 17

### Dynamic Adsorption Test—Gas Stream Purification

The capability of the adsorbents of the invention to purify olefin monomers and cracking gases was proven in a dynamic test for butadiene recovery from propylene flow. The test unit included:
a stainless steel microreactor (0.6×10 cm);
two gas probe injectors (inlet and outlet microreactor);
Teflon receiver for outgoing gas of 50 cm³ volume;
a gas chromatograph with flame ionization detector (FID).

Initial gas mixture was prepared in a 10 L-gas cylinder and had the following gas composition: propylene—30.0; nitrogen—70.0% v.; 1,3-butadiene—15 ppm.

The portion of the crushed adsorbent of Example 8 in the amount of 100 mg and with particle size of 0.25-0.50 mm was placed in the central part of microreactor. Remaining free volume of the microreactor was loaded by crushed quartz with granulation of 1-2 mm. The adsorbent sample was preliminarily trained at 250-270° C. in nitrogen flow for 2.5 hours, then cooled down to ambient temperature, and gas mixture fed the microreactor by means of a 50 ml-syringe. Gas feeding was carried out at ambient temperature and atmospheric pressure by short pulses of 34-35 ml volume with a flow rate of 1 L/min. Interruption between successive pulses—3-5 min. The total number of pulses over the sample test is >20.

FIG. 2 demonstrates a chromatogram of the product gas on the 21^{th} pulse alongside initial gas mixture. Therewith a small peak on the tail of the large one is 1,3-butadiene in the amount of 15 ppm in initial gas mixture (FIG. 2 b), which completely disappears in the purified gas (FIG. 2 a). Total amount of butadiene contaminated in all injected pulses allowed to estimate dynamic adsorption capacity of the adsorbent of the present invention. The obtained value of >0.36% w. should be recognized as prominent taking into account an extremely low partial pressure of the adsorbate, unusually high for adsorption technologies linear velocity of 0.6 m/sec, and a negligible contact time of ˜10 msec. And what is the most important for commercial applications that the test results confirm the possibility to reach practically complete removal of dienes from monomers on the level <1 ppm with the use of the adsorbents of the present invention.

### EXAMPLE 18

### Dynamic Test—Flue Gas Purification

The purpose of this test consists of providing proof of applicability of the adsorbents of the present invention to flue and waste gases purification. The experimental procedure, technique and test conditions were very much the same as in Example 17. The gas composition simulated combustion products or flue gases and had the following formulation: air—77.0; carbon dioxide—9.0; carbon monoxide—4.0; methane—3.3; hydrogen—3.5; moisture—3.2% v.; 1,3-butadiene—25 ppmv.

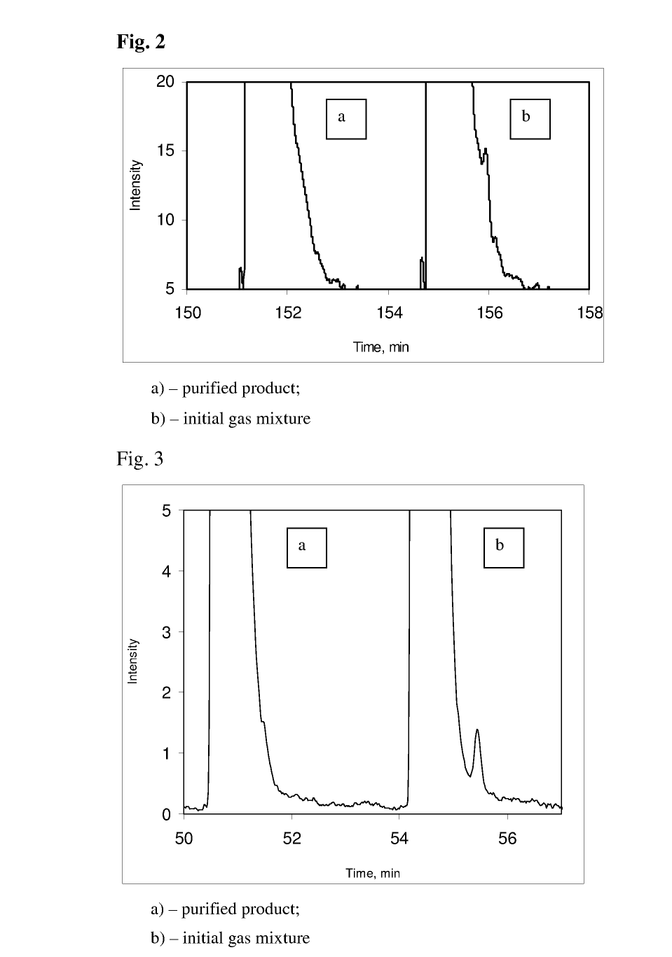

Adsorbents of Examples 8 and 11 were employed in the tests. Altogether 11 pulses of gas mixture were made in each case. FIG. 3 compares analysis of feed and product gas outlet adsorbent bed of Example 8 in the 11^{th} pulse. Herein a small peak on the tail of the large one is 1,3-butadiene in the amount of 25 ppm inlet the adsorbent bed (chromatogram b), while chromatogram (a) shows butadiene content in the product gas only in trace amounts.

Combined report for all pulse tests is given in Table 4.

TABLE 4
1,3-Butadiene Concentration Outlet Adsorbents in Successive Pulses

| | Butadiene Content in Product Gas, ppm | | | | | | | | | | |
|---|---|---|---|---|---|---|---|---|---|---|---|
| | Pulse No | | | | | | | | | | |
| Adsorbent | 1 | 2 | 3 | 4 | 5 | 6 | 7 | 8 | 9 | 10 | 11 |
| Example 8 | 0 | 0 | 0 | 0 | 0 | 0 | 0 | 0 | 0 | 0 | <1 |
| Example 11 | 0 | 0 | 0 | 0 | 0 | 0 | 0 | 0 | 0 | <1 | <2 |

The data in Table 4 manifest outstanding performance of the adsorbents of the invention for butadiene removal. The first 9 pulses gave an absolutely identical picture—complete removal of butadiene from gas stream. Analysis of the product gas in the 10^{th} and 11^{th} pulses was repeated at least 3 times. Butadiene traces in quantity of <1 ppm appear in the 10^{th} pulse and <2 ppm in the 11^{th} pulse for the adsorbent of Example 11 and <1 ppm in the 11^{th} pulse for the adsorbent of Example 8. Thus, even in the 11^{th} pulse the nano-structured adsorbent of the invention allows recovery >95% of 60 μg (25 ppmv) butadiene from gas stream.

Therefore, the test results prove a low sensitivity of the adsorbents of the present invention to the high content of moisture, hydrogen and carbon monoxide in purified gas. In such a manner, adsorbents of the invention are quite suitable for flue, waste and automotive gases purification. Moreover, high adsorption capacity and complete butadiene removal in the first 9 pulses show that the adsorbents of the invention may attain any required degree of pollutant recovery up to ppb level by proper variation of the adsorbent bed volume and gas flow rate.

### EXAMPLE 19

### Adsorbent Regenerability Test

Adsorbent of Example 2 after 1 run of hexene-1 purification in the dynamic test of Example 14 was rinsed with ˜250 ml of pure hexene-1 at ambient temperature and flow rate of adsorption. The adsorbent bed then was fed with hydrogen flow at a rate of 100 ml/min and a gradually raised temperature from ambient up to 180° C. Regeneration time comprised 3.5 hours. Then, adsorbent was cooled down and test operating procedure of Example 14 for 1,3-butadiene removal from hexene-1 flow was repeated.

In all, 30 adsorption-regeneration cycles were carried out. As this took place, butadiene dynamic adsorption capacity remained unchanged on the level of 1.9-2.3% w.

The adsorbent sample exposed in 30 adsorption-regeneration cycles was analyzed for carbonaceous deposits accumulation. Coke content in the sample was determined as high as 0.03% w using the Kahn balance technique by means of combustion in airflow at a rate of 30 ml/min and temperature 500° C.

Thus, the test's outcome supports complete regenerability of the adsorbent of the present invention and high efficacy of the process, according to the invention, for adsorbent regeneration from adsorbed dienes. At the same time, taking into account that perceptible decline of adsorption capacity of zeolite adsorbents usually begins after accumulation of ˜2% w carbonaceous deposits, absolutely negligible coke content in the adsorbent having passed 30 adsorption-regeneration cycles permits an assured prediction of the adsorbent long-life operation in various existing and newly developed processes for dienes removal.

Therefore, the above examples demonstrate that the adsorbents of the invention and processes for dienes removal therewith are highly reliable and efficient in liquid and gas streams purification and provide essential technical and economical benefits in many commercial applications. Furthermore, incorporation of transition metal polycations into synthetic faujasites structure leads to a number of advantages of diene adsorbents of the present invention over prior art adsorbents and corresponding purification processes. These are:
An enhanced adsorption capacity at very low diene concentrations and partial pressures;
Capability to provide a profound purity of purified liquid and gas streams with residual dienes content below 500 and 1500 ppb respectively with an adequate upgrade of petrochemical products quality and reduction of operation costs for their processing;
A very low susceptibility to the presence of moisture, sulfur and nitrogen-contaminated compounds and ability to purify highly moisturized and polluted liquid and gas streams without additional investments;
Applicability and high efficiency of the adsorbent for purification of flue, waste and exhaust gases of diverse composition;
Reduced cost of the adsorbent due to lack of noble metals in its composition and necessity of extremely high cation exchange degrees along with a simple, easy to commercialize adsorbent manufacturing procedure;
Complete regenerability and full reproducibility of original diene adsorption capacity over multiple adsorbent regeneration operations resulting in highly economic technologies for liquid and gas streams purification with a simplified process flow sheet.

Although the invention has been described with reference to specific examples, these experiments are merely exemplary and variations are contemplated. For instance, the adsorbents can comprise other combinations of transition metal cations and faujasites than those illustrated in the examples. The adsorbents can be prepared by other techniques, such as chemical vapors deposition, alkaline treatment of conventionally exchanged zeolites or solid state exchange. Similarly, dienes removal processes may include various combinations of adsorption and regeneration steps. Furthermore, the adsorbents of the invention can be used for dienes removal from many other liquids and gases including non-hydrocarbon origination.

Thus, the scope of the invention is limited only by the breadth of the appended claims and their legal equivalents.

We claim:

1. An adsorbent for dienes containing single or multiple transition metal ion-exchanged forms of faujasites with a silicon to aluminum ratio from about 0.9:1 to about 9.0:1, heretofore at least part of transition metal ions comprises nano-structured polycations of a general formula [Tr_{α}O_{β}]ⁿ⁺, wherein Tr means transition metal and wherein a varies from 2 to 8, β—from 0 to 4, and n—from 1 to 3.

2. The adsorbent of claim 1 wherein said transition metals include IB. IIB, VIIB and VIII Group metals preferably selected from the group consisting of copper, cadmium, zinc, manganese, nickel and iron.

3. The adsorbent of claim 1 further comprising balanced cations of alkali and/or alkaline-earth metals selected from the group consisting of sodium, potassium, calcium and magnesium.

4. The adsorbent of claim 1 wherein said single or multiple transition metal ion-exchanged faujasites have a general formula:
(Tr₁M⁺)ₘ[Tr₁M_{α}O_{β}]ₙ⁺(Tr₂M²⁺)ₚ[Tr₂M²⁺_{γ}O_{δ}]_{q}²⁺(Tr₃M)_{c}[Tr₃M_{ε}O_{ν}]_{d}(Me⁺)ₑ(Me²⁺)_{f}Si_{σ}Al_{τ}O_{φ}
wherein Tr₁M, Tr₂M, Tr₃M mean mononuclear mono-, bi-, and trivalent transition metal cations and [Tr₁M_{α}O_{β}]⁺, [Tr₂M_{γ}O_{δ}]²⁺, [Tr₃M_{ε}O_{ν}]³⁼ mean corresponding polycations, wherein n, q and d comprise Tr₁M, Tr₂M and Tr₃M polycation exchange degrees, wherein α, γ and ε comprise numbers of TrM atoms in polycations that vary from 2 to 8 and β, δ, and ν are numbers of oxygen atoms in TrM polycations that vary from 0 to 4, wherein Me⁺, Me²⁺ mean monocations of alkali and alkaline-earth metals and m, p, c, e, f comprise corresponding ion exchange degrees and wherein σ/τ comprises silicon to aluminum ratio that varies from 0.9 to 9.0.

5. The adsorbent of claim 1 wherein said faujasites comprise a low-silica faujasite LSF having a silicon to aluminum ratio from about 0.9:1 about 1.1:1.

6. The adsorbent of claim 1 wherein said faujasites comprise a synthetic faujasite X having a silicon to aluminum ratio from about 1.25:1 to about 1.75:1.

7. The adsorbent of claim 1 wherein said faujasites present a synthetic faujasite Y having a silicon to aluminum ration from about 1.8:1 to about 2.5:1.

8. The adsorbent of claim 1 wherein said faujasites are dealuminated or ultrastable faujasites Y having a silicon to aluminum ratio from about 2.75:1 to about 9.0:1.

9. An adsorbent for dienes removal from moisturized liquid and gas streams comprising mono- and polynuclear transition metal ion-exchanged forms of dealuminated or ultrastable faujasites with a silicon to aluminum ratio from about 3:7:1 to about 5.0:1.

10. The adsorbent of claims 1 and 9 wherein content of said transition metal polycations is between about 1.5% and about 12.0% w.

11. A process for purifying diene-contaminated liquid and gas streams which comprises passing a feed stream over the bed of the adsorbent of claim 1 whereby a predominant part of diene impurity is removed by the adsorbent.

12. The process of claim 11 wherein said dienes mainly comprise C₄-C₆ diolefins further including butadienes, pentadienes and hexadienes.

13. The process of claim 11 wherein content of dienes in said liquid and gas streams is in the range from about 10 ppm to about 500 ppm.

14. The process of claim 11 for purifying liquid streams wherein the residual content of dienes in liquid stream outlet the adsorbent bed is from about 200 ppb to about 500 ppb.

15. The process of claim 11 for purifying gas streams wherein the residual content of dienes in the gas stream outlet the adsorbent bed is from about 500 to about 1500 ppb.

16. The process of claim 11 for purifying liquid streams wherein the liquid feed stream is passed over the adsorbent bed at a temperature from about 0 to about 60° C., pressure from about atmospheric to about 60 bar and liquid hourly space velocity from about 0.1 to about 10 h⁻¹.

17. The process of claim 11 for purifying gas streams wherein the gas feed stream is passed over the adsorbent bed at a temperature from about 0 to about 100° C., pressure from about atmospheric to about 150 bar and linear velocity from about 0.01 to about 0.4 m/sec.

18. The process of claim 11 further comprising a periodic regeneration of the adsorbent by rinsing thereof with a part of purified flow at ambient temperature and heating in a flow of hydrogen or hydrogen-contaminated gas at a temperature from about 150 to about 200° C.
